(19) United States
(12) Patent Application Publication
(10) Pub. No.: US 2008/0004635 A1
(43) Pub. Date: Jan. 3, 2008

(54) HAIR REMOVAL APPLIANCE AND METHOD OF USING SAME

(76) Inventors: Anna McMillan, Huntersville, NC (US); Shawn McMillan, Huntersville, NC (US)

Correspondence Address: ADAMS EVANS P.A., Suite 2350 Charlotte Plaza, 201 South College Street, CHARLOTTE, NC, 28244, US

(21) Appl. No.: 11/424,606

(22) Filed: Jun. 16, 2006

(51) Int. Cl. A61B 17/50

(52) U.S. Cl. 606/134

(57) ABSTRACT

An appliance for aiding in the removal of a hardened wax from a person's body includes a curved member defining a handle for being grasped by a user and a substantially flat spatula for positioning on a person's skin. Cavities are formed in the spatula for receiving a wax in an initially liquid state and allowing the wax to harden within the cavity so that the wax becomes attached to the appliance. The cavities can be linear grooves formed in the spatula, or holes that penetrate all the way through the spatula.

### TECHNICAL FIELD AND BACKGROUND OF THE INVENTION

The invention relates to a tool for aiding in the removal of a solidified substance from a surface. The invention is particularly suited for facilitating the removal of hardened wax from a person's skin in a hair removal process.

A variety of methods have been developed for removing unwanted hair from various parts of the human body. One typical hair removal method is “waxing”, a process by which wax is used to pull unwanted hair out by the roots. Warm wax in liquid form is applied to the desired area of the body, and is allowed to cool and harden on the hair to be removed. The wax is then peeled off of the body, removing the unwanted hair with it.

While waxing can be an effective method for removing hair, it is generally painful and uncomfortable. One factor contributing to this discomfort is that before the entire mass of hardened wax can be peeled off, the person applying the wax must initially get a firm grasp of some portion of the hardened wax. This generally involves using the fingernails to pick at an edge of the hardened wax and peel away a small portion of the hardened wax from the skin so that it can be held with the fingers. This can be cumbersome and time consuming for the technician applying the wax, and uncomfortable for the person undergoing the wax treatment. In addition, it can be unsanitary, and requires the use of both hands of the technician. Furthermore, this discomfort immediately precedes and therefore exacerbates the pain associated with the completely peeling away the wax.

In an attempt to eliminate this undesirable aspect of waxing, some technicians place a layer of material, such as a cloth on top of the cooling wax. As the wax cools it adheres to the cloth, so that the technician can pull the cloth to remove the wax. As such, the technician does not have to pick at the edge of the wax with the fingernails in order to peel it off. While a cloth can be used in this manner with some waxes, such as “soft” waxes for removing relatively fine hair, it is not suitable for all waxes. For example, the cloths do not adhere well to the “hard” waxes designed for removing relatively coarse hair. In addition, cloths that initially adhere to the wax may pull off of the wax or tear during the peeling process.

In an effort to overcome and eliminate the aforementioned problems, the present invention was conceived.

### SUMMARY OF THE INVENTION

Therefore it is an object of the present invention to provide an appliance that facilitates the removal of wax from a person's body.

It is another object of the invention to provide a hair removal appliance that can be used with a variety of waxes, including hard waxes for coarse hair.

These and other objectives of the present invention are achieved by providing a tool that can aid in the removal of a solidified substance from a surface. In particular, the invention provides an appliance for aiding in the removal of a hardened wax from a body having a handle for being grasped by a user and a spatula for being positioned on the body. At least one cavity is formed in the spatula for receiving a wax in an initially liquid state and allowing the wax to harden within the cavity so that the wax becomes attached to the appliance. The term “cavity”, as used throughout this application, refers generally to all kinds of voids, regardless of shape or size. In addition, the cavity may or may not penetrate all the way through the spatula. The term “body” refers generally to any and all parts of the body of a human or animal.

According to a preferred embodiment of the invention, the wax is attached to the appliance and removed from the body by grasping and pulling the handle.

According to another preferred embodiment of the invention, the handle and the spatula are integrally formed from a single curved member.

According to yet another preferred embodiment of the invention, the handle and the spatula are integrally formed from a single angled member, and the handle is angled in relation to the spatula.

According to yet another preferred embodiment of the invention, a plurality of linear grooves are formed in the upper surface of the spatula. The linear grooves do not penetrate all the way through the spatula.

According to yet another preferred embodiment of the invention, the linear grooves are transverse to the spatula.

According to yet another preferred embodiment of the invention, a plurality of through holes are formed in the spatula.

According to yet another preferred embodiment of the invention, the appliance has a polygonal shape, and increases gradually in width from the handle to the spatula.

According to yet another preferred embodiment of the invention, the spatula has a lower surface for being positioned on the body, and an upper surface opposite the lower surface. A plurality of linear grooves are formed in the upper surface.

According to yet another preferred embodiment of the invention, the appliance includes a generally oval shaped layer of material having an edge curved such that the edge extends over the upper surface of the spatula to form the handle.

According to yet another preferred embodiment of the invention, the spatula has a lower surface opposite the upper surface for being positioned on the body, and a plurality of through holes are formed in the spatula.

According to yet another preferred embodiment of the invention, the appliance is made of plastic, clay and/or wood.

According to yet another preferred embodiment of the invention, the spatula is contoured to conform to the body.

According to yet another preferred embodiment of the invention, a hair removal appliance for aiding in the removal of a hardened wax from a body includes an outer member defining an interior area, a first end curved upward in relation to the rest of the outer member to form a handle for being grasped by a user, and a second substantially flat end for being positioned on the body. An inner member is connected to the outer member and positioned within the interior area defined by the outer member. The inner member and the second end of the outer member define an opening for receiving liquid wax and allowing the wax to harden so that the wax becomes attached to the appliance and removal of the appliance from the body simultaneously removes the wax from the body.

According to yet another preferred embodiment of the invention, the outer member is substantially oval shaped.

According to yet another preferred embodiment of the invention, a method of removing hair from a body includes providing an appliance having a handle and a spatula with at least one cavity formed therein. The appliance is positioned such that the spatula lies on the body and is adjacent a desired area of the body from which hair is desired to be removed. A liquid wax is applied to the desired area of the body and the spatula, and a portion of the liquid wax on the spatula is received within the cavity and hardens therein. After the wax has hardened, the appliance is removed from the body so that the hardened wax and hair attached to the wax are removed from the body.

According to yet another preferred embodiment of the invention, the handle is grasped and the appliance is pulled away from the body.

According to yet another preferred embodiment of the invention, the appliance is positioned such that the spatula covers an area of the body on which hair is to remain.

According to yet another preferred embodiment of the invention, a user grasps the handle with a first hand, and can maintain the skin taut with a second hand.

### BRIEF DESCRIPTION OF THE DRAWINGS

Some of the objects of the invention have been set forth above. Other objects and advantages of the invention will appear as the invention proceeds when taken in conjunction with the following drawings, in which:

FIG. 1 is a perspective view of a hair removal appliance according to a preferred embodiment of the invention;

FIG. 2 is a side elevation of the hair removal appliance of FIG. 1;

FIG. 3 is an environmental view illustrating a method of using the appliance of FIG. 1 according to a preferred embodiment of the invention;

FIG. 4 is another environmental view illustrating a method of using the appliance of FIG. 1 according to a preferred embodiment of the invention;

FIG. 5 is yet another environmental view illustrating a method of using the appliance of FIG. 1 according to a preferred embodiment of the invention;

FIG. 6 is an environmental view illustrating another method of using the appliance of FIG. 1 according to a preferred embodiment of the invention;

FIG. 7 is another environmental view illustrating a method of using the appliance of FIG. 1 according to a preferred embodiment of the invention;

FIG. 8 is yet another environmental view illustrating a method of using the appliance of FIG. 1 according to a preferred embodiment of the invention;

FIG. 9 is a perspective view of a hair removal appliance according to another preferred embodiment of the invention;

FIG. 10 is a side elevation of the appliance of FIG. 9;

FIG. 11 is an environmental view illustrating a method of using the appliance of FIG. 9 according to a preferred embodiment of the invention;

FIG. 12 is another environmental view illustrating a method of using the appliance of FIG. 9 according to a preferred embodiment of the invention;

FIG. 13 is yet another environmental view illustrating a method of using the appliance of FIG. 9 according to a preferred embodiment of the invention;

FIG. 14 is a perspective view of a hair removal appliance according to yet another preferred embodiment of the invention;

FIG. 15 is a side elevation of the appliance of FIG. 14;

FIG. 16 is an environmental view illustrating a method of using the appliance of FIG. 14 according to a preferred embodiment of the invention;

FIG. 17 is another environmental view illustrating a method of using the appliance of FIG. 14 according to a preferred embodiment of the invention; and

FIG. 18 is yet another environmental view illustrating a method of using the appliance of FIG. 14 according to a preferred embodiment of the invention.

### DESCRIPTION OF THE PREFERRED EMBODIMENT AND BEST MODE

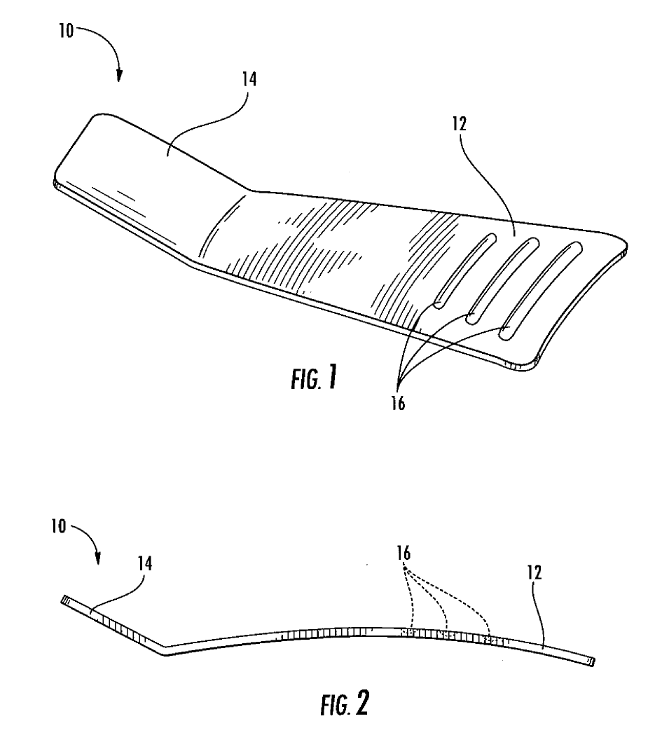

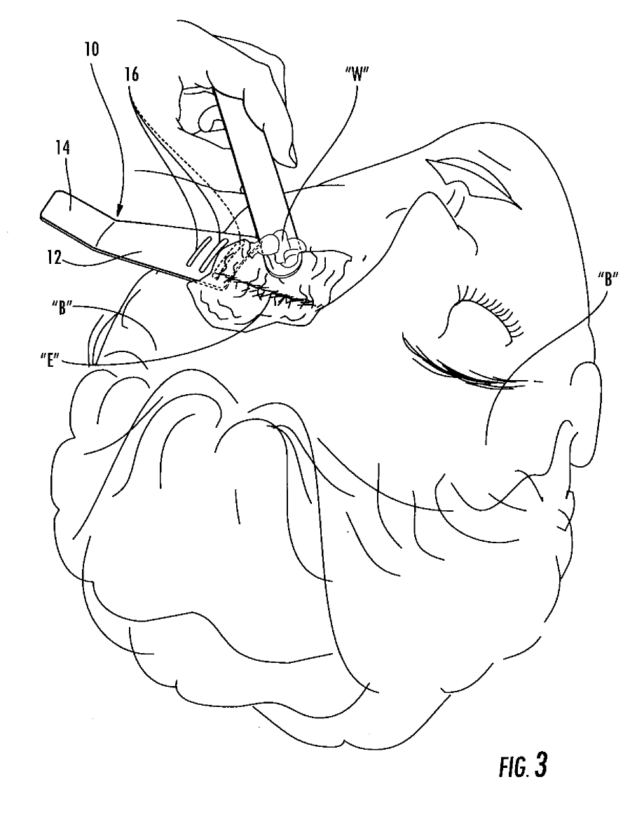

Referring now specifically to the drawings, a hair removal appliance according to a preferred embodiment of the present invention is illustrated in FIGS. 1 and 2, and shown generally at reference numeral 10. The appliance 10 is angled, and includes a spatula 12 with a handle 14 extending slightly upwardly from the spatula 12. As shown in FIG. 2, the spatula 12 is contoured to conform to an area of the body “B” from which hair is to be removed. Alternatively, the spatula 12 can be completely flat.

The spatula has an upper surface on which wax “W” is applied, and a lower surface that is placed on the body “B”. A plurality of linear grooves 16 are formed in the upper surface of the spatula 12, as shown in FIG. 1. The grooves 16 do not penetrate all the way through the spatula 12. Preferably, the spatula 12, handle 14 and grooves 16 are all integrally formed from a single piece of suitable material, such as plastic, clay or wood.

The spatula 12 can include an adhesive so that the appliance 10 can stick to the body without the user holding the handle 14. This frees up one of the user's hands to do other things, such as holding the skin taut while the other hand applies the wax “W”. The adhesive can be in the form of a strip that is affixed to the lower surface of the spatula 12, and has a removable cover that is peeled off immediately before use.

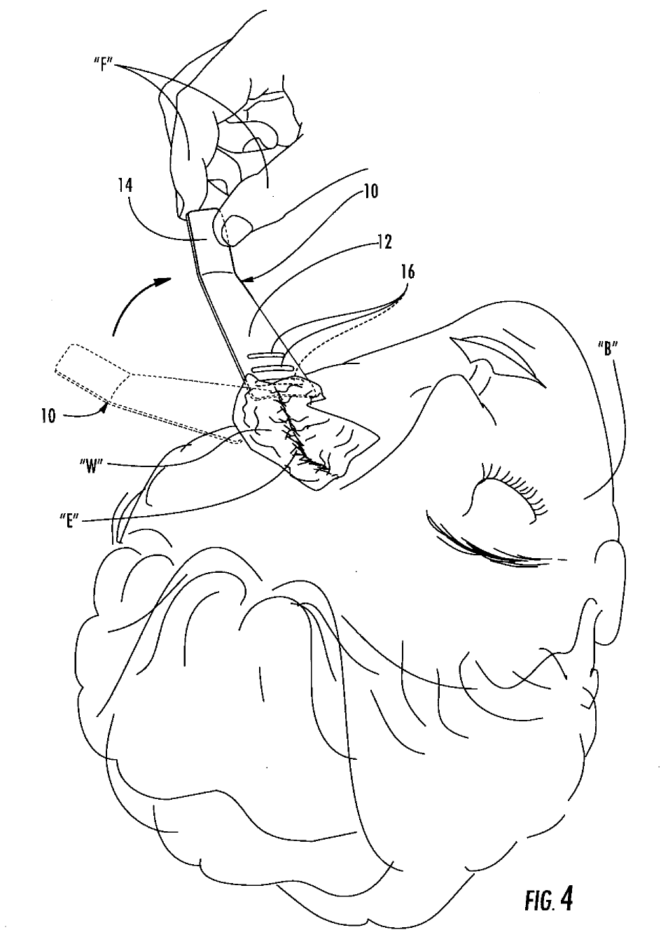

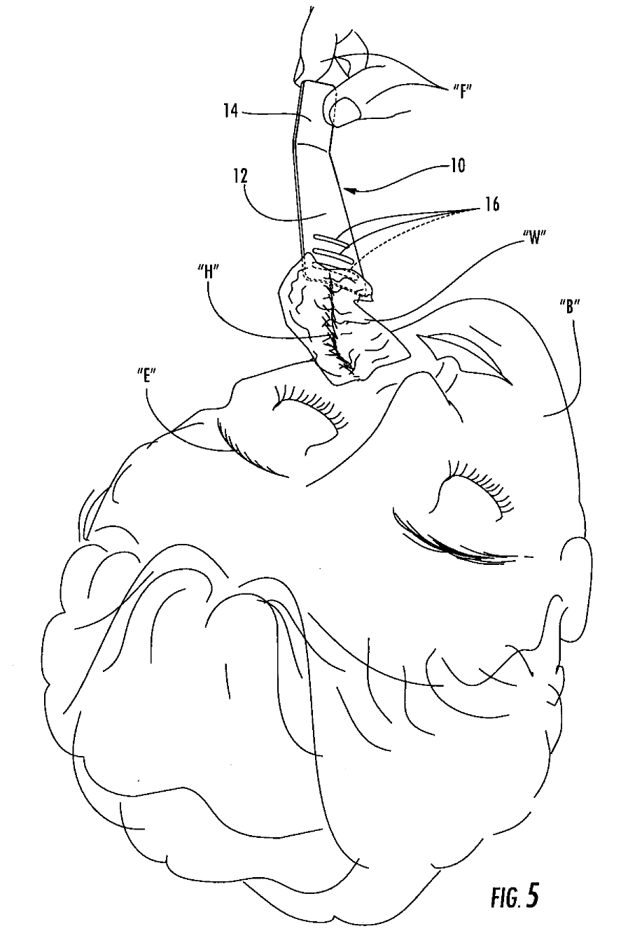

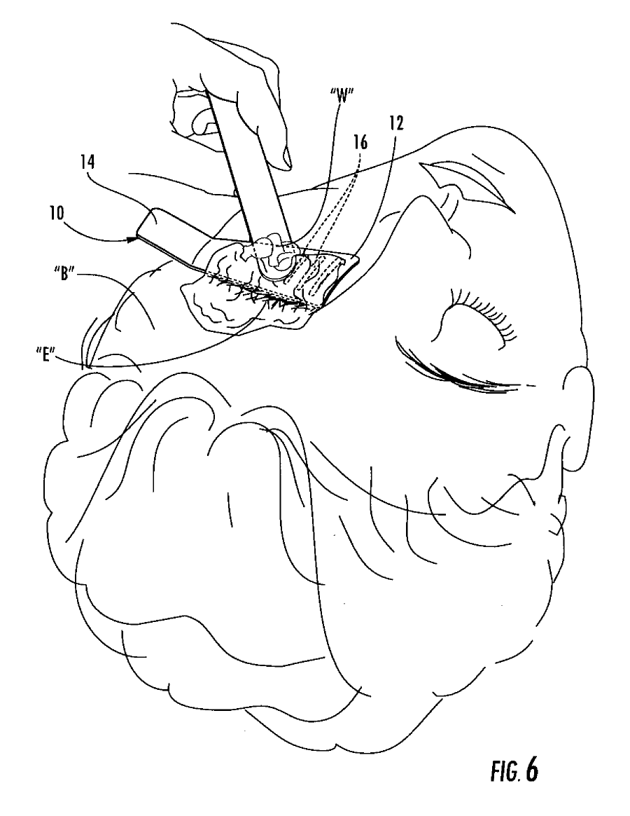

FIGS. 3-5 illustrate a preferred method of using the appliance 10. The appliance 10 is positioned such that the spatula 12 lies proximate the desired area of the person's body “B” that is to undergo hair removal, such as a portion of the eyebrows “E”. As shown in FIG. 3, warm liquid wax “W” of the type used for hair removal is applied to the desired portion of the person's eyebrow “E” and over the upper surface of the spatula 12. Some of the wax “W” seeps into grooves 16 in the appliance 10. The wax “W” is left on for about one minute to cool and harden so that it firmly attaches to the hair “H” to be removed. Wax “W” within the grooves 16 also cools and hardens, resulting in a firm attachment between the wax “W” and the appliance 10. When the wax “W” has sufficiently hardened, the user grasps the handle 14 with the fingers “F” and quickly pulls the appliance 10 away from the person's body “B”, as shown in FIG. 4. The hardened wax “W”, which has attached itself to the hair “H” and the spatula 12, is removed from the person's body “B” resulting in the fast and efficient removal of unwanted hair “H”, as shown in FIG. 5.

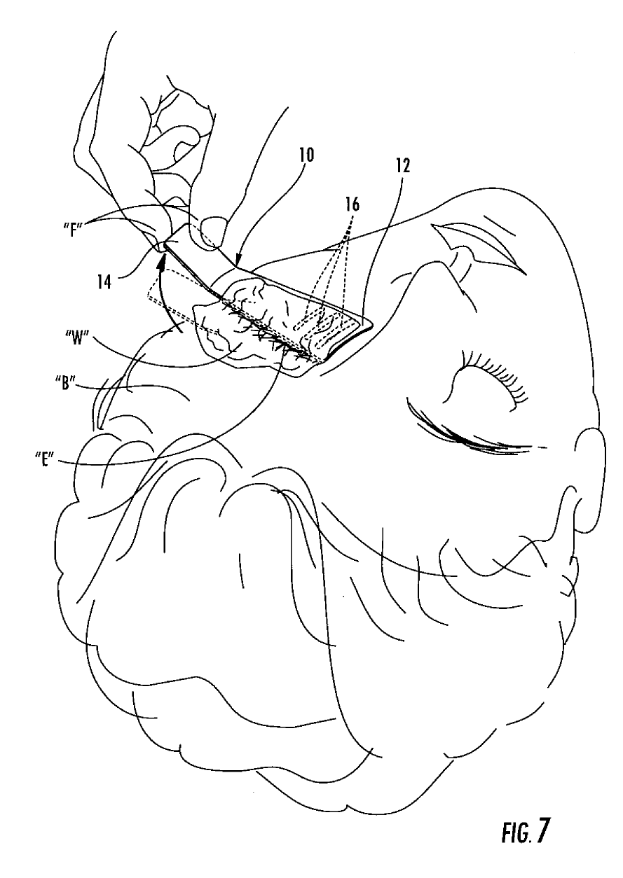

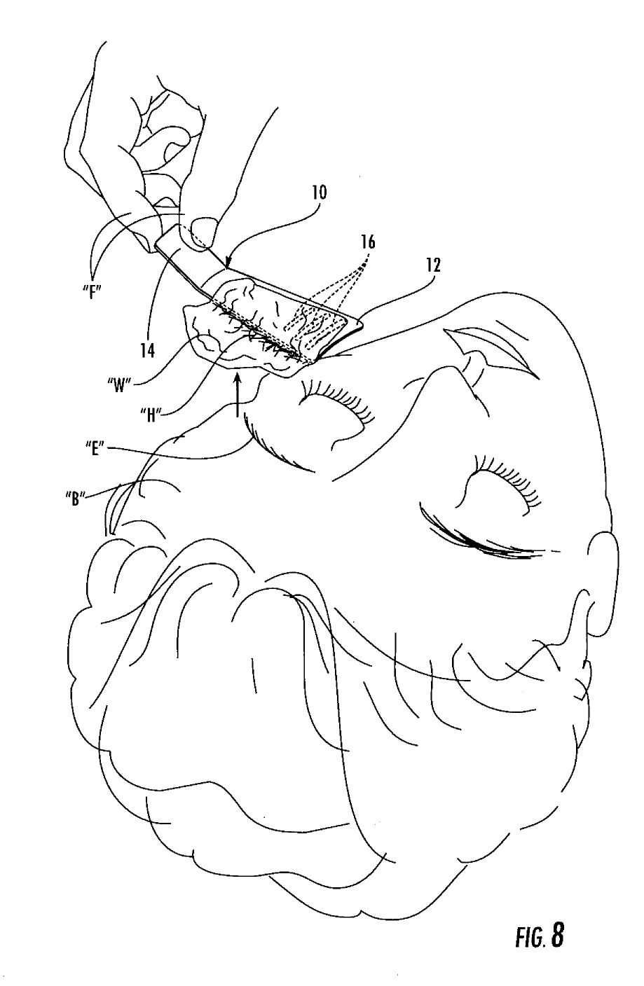

In addition to facilitating the removal of the hardened wax “W”, the appliance 10 can provide a cover to prevent wax “W” from getting on adjacent areas of the body “B” on which hair is to remain. The appliance 10 is positioned on the person's body such that the spatula 12 covers an area from which hair is not to be removed. As such, the appliance 10 can be used to remove specific hairs “H” while leaving a neat and even line of hair intact on the body. For example, the appliance 10 is particularly useful in precisely shaping eyebrows “E”, as illustrated in FIGS. 6-8.

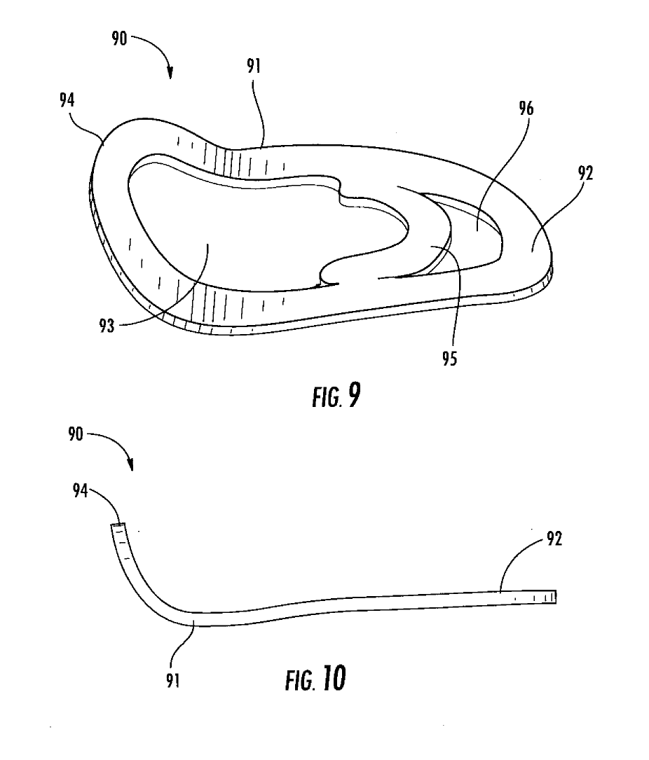

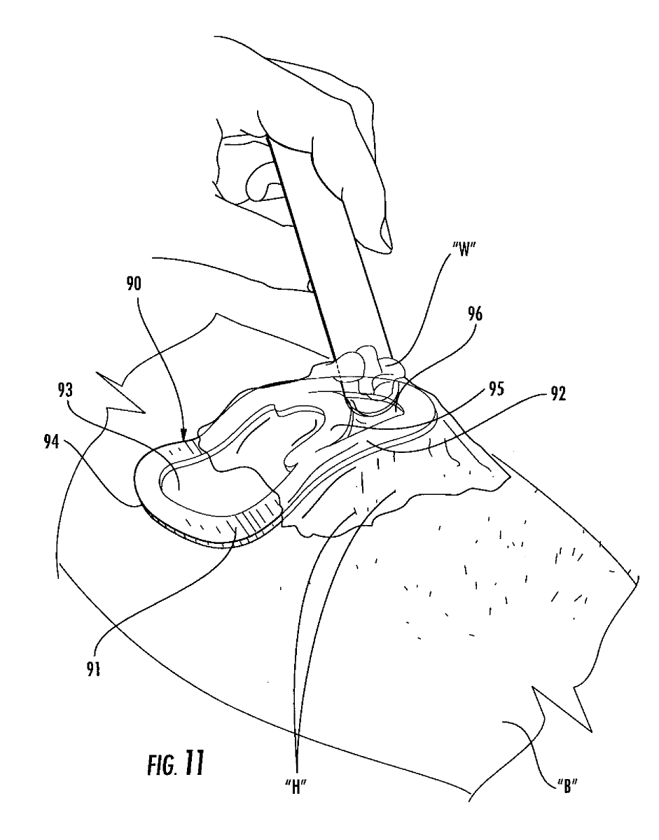

A hair removal appliance according to another preferred embodiment of the invention is illustrated in FIGS. 9 and 10, and shown generally at reference numeral 90. The appliance 90 comprises a generally oval shaped outer member 91 defining an interior area 93, and an inner member 95 positioned within the interior area 93 and connected to the outer member 91. The outer member 91 and inner member 95 can be integrally formed from a single piece of material, or can be separate pieces joined together such as with an adhesive. A generally flat section of the outer member 61 and the inner member 95 comprise a spatula 92. An upwardly curved portion of the outer member 91 defines a handle 94. The outer member 91 and inner member 95 define an opening 96 in the spatula 92.

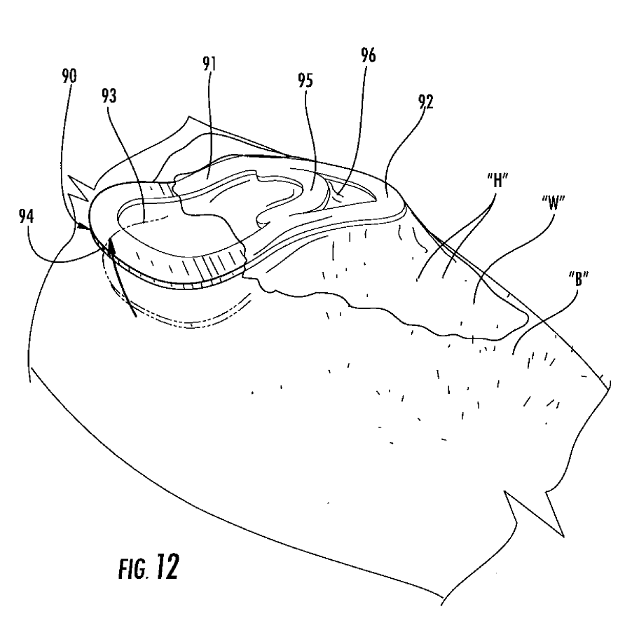

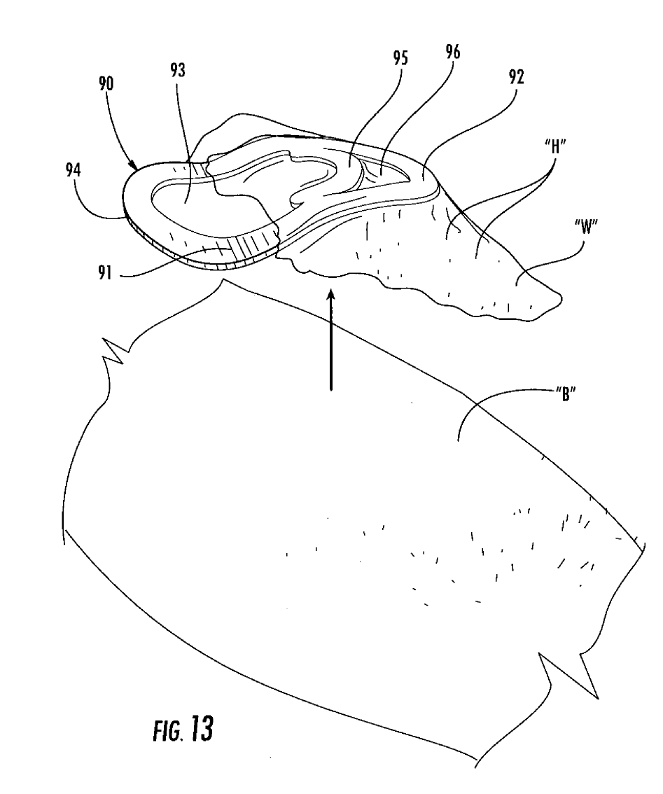

FIGS. 11-13 illustrate a preferred method of using the appliance 90. The appliance 90 is positioned such that the spatula 92 lies proximate the desired area of a person's body “B” from which hair is to be removed. As shown in FIG. 11, warm liquid wax “W” is applied to the desired area of the body “B” and over the upper surface of the spatula 92. Some of the wax “W” is applied to the opening 96 defined by the outer and inner members 91, 95 of the appliance 90. The wax “W” is allowed to cool and harden so that it firmly attaches to the hair “H” that is to be removed. Wax “W” within the opening 96 also cools and hardens resulting in a firm attachment between the wax “W” and the appliance 90. When the wax “W” has sufficiently hardened, the user grasps the handle 94 of the appliance 90, and quickly pulls the appliance 90 away from the person's body “B”, as shown in FIG. 12. The hardened wax “W”, which has attached itself to the hair “H” and the appliance 90, is removed from the person's body “B” resulting in the fast and efficient removal of unwanted hair “H”, as shown in FIG. 13.

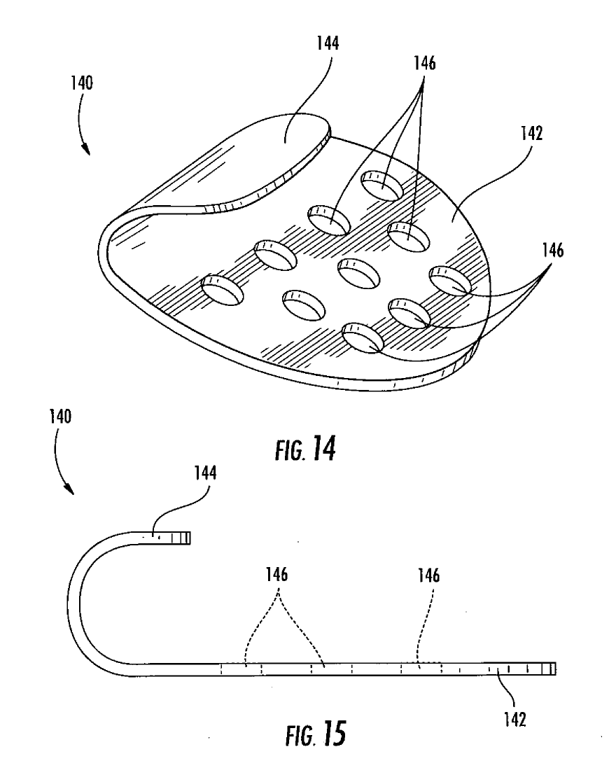

Yet another hair removal appliance is illustrated in FIGS. 14 and 15, and shown generally at reference numeral 140. The appliance 140 is generally oval shaped, and has a substantially flat spatula 142 and a handle 144 extending upwardly and slightly over the upper surface of the spatula 142. A plurality of holes 146 are formed in the upper surface of the spatula 142, as shown in FIG. 14.

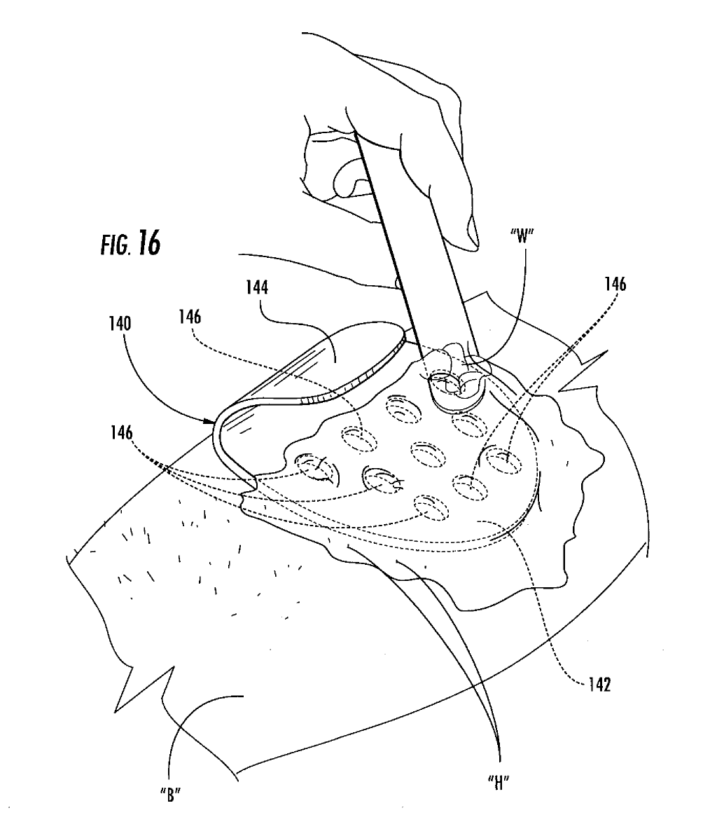

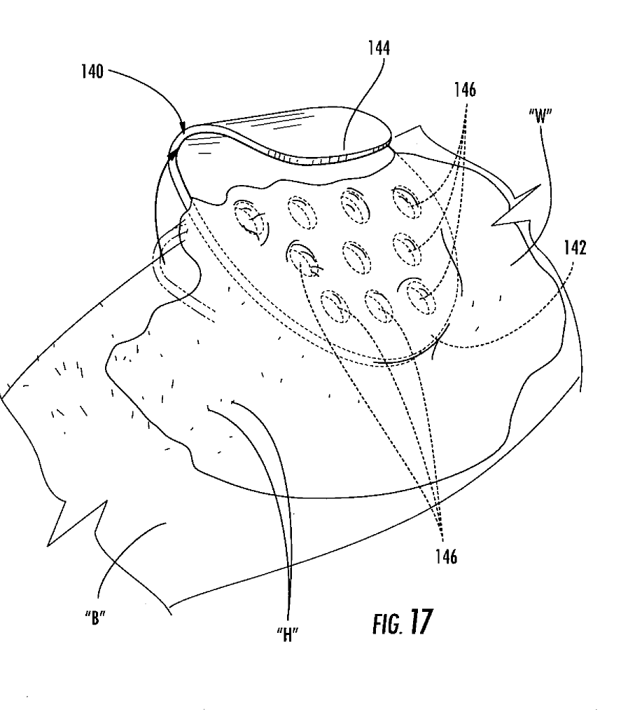

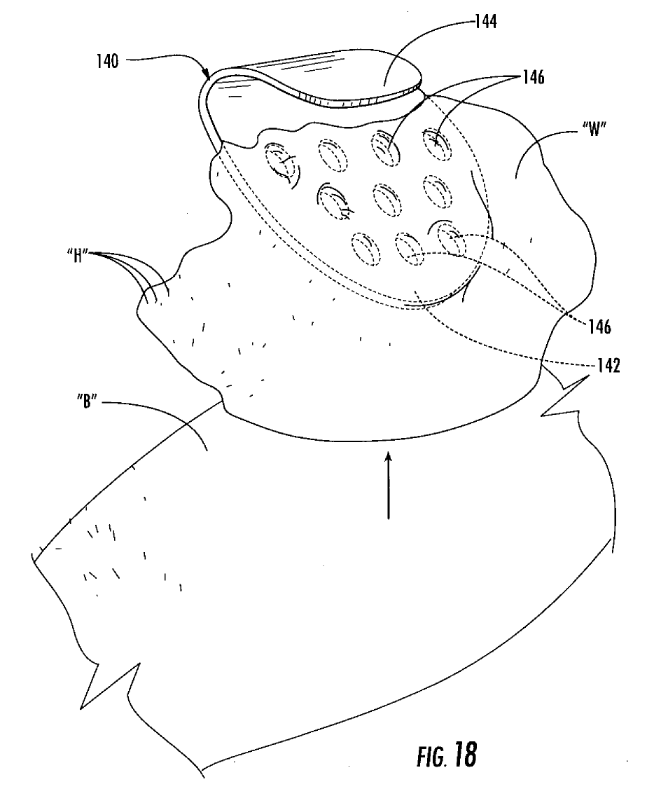

FIGS. 16-18 illustrate a preferred method of using the appliance 140. The appliance 140 is positioned such that the spatula 142 lies proximate the desired area of a person's body “B” that is to undergo hair removal. As shown in FIG. 16, warm liquid wax “W” is applied to the desired portion of the person's body “B” and over the upper surface of the spatula 142. Some of the wax “W” seeps into holes 146 in the appliance 140. The wax “W” is allowed to cool and harden so that it firmly attaches to the hair “H” that is to be removed. Wax “W” within the holes 146 also cools and hardens resulting in a firm attachment between the wax “W” and the appliance 140. When the wax “W” has sufficiently hardened, the user grasps the handle 144, and quickly pulls the appliance 140 away from the person's body “B”, as shown in FIG. 17. The hardened wax “W”, which has attached itself to the hair “H” and the spatula 142, is removed from the person's body “B” resulting in the fast and efficient removal of unwanted hair “H”, as shown in FIG. 18.

The appliances 10, 90, 140 described above can be made with a polymer clay sold under the trade name SCULPEY by Polyform Products Co., Elk Grove, Ill. The clay is shaped into the desired form and covered in tin foil. It is then cured in an oven at about 275 degrees Fahrenheit for about fifteen minutes. Alternatively, the appliances 10, 90, 140 can be molded from plastic or wood.

A hair removal appliance and method for using same are disclosed above. Various embodiments of the invention can be made without departing from its scope. For example, it is contemplated that the invention can be used in applications other than hair removal waxing. Furthermore, the foregoing description of the preferred embodiments of the invention and the best mode for practicing the invention are provided for the purpose of illustration only and not for the purpose of limitation the invention being defined by the claims.

1. A hair removal appliance for aiding in the removal of a hardened wax from a body, comprising a handle for being grasped by a user and a spatula for positioning on a body adjacent an area of hair removal having at least one cavity formed in the spatula for receiving a wax in an initially liquid state and hardening within the cavity whereby wax becomes attached to the appliance.

2. An appliance according to claim 1, wherein the wax attached to the appliance can be removed from the body by grasping and pulling the handle.

3. An appliance according to claim 1, wherein the handle and the spatula are integrally formed from a single curved member.

4. An appliance according to claim 1, wherein the handle and the spatula are integrally formed from a single angled member, and the handle is angled in relation to the spatula.

5. An appliance according to claim 1, wherein the at least one cavity comprises a plurality of linear grooves formed in an upper surface of the spatula.

6. An appliance according to claim 5, wherein the linear grooves are transverse to the spatula.

7. An appliance according to claim 1, wherein the at least one cavity comprises a plurality of through holes formed in the spatula.

8. An appliance according to claim 1, wherein the appliance has a polygonal shape, and increases gradually in width from the handle to the spatula.

9. An appliance according to claim 8, wherein the spatula defines a lower surface for being positioned on the body and an upper surface opposite the lower surface, and further wherein the at least one cavity comprises a plurality of linear grooves formed in the upper surface.

10. An appliance according to claim 1, wherein the appliance comprises a generally oval shaped layer of material having an edge curved such that the edge extends over an upper surface of the spatula to form the handle.

11. An appliance according to claim 10, wherein the spatula defines a lower surface opposite the upper surface for being positioned on the body, and further wherein the at least one cavity comprises a plurality of through holes formed in the spatula.

12. An appliance according to claim 1, wherein the appliance comprises at least one selected from the group consisting of plastic, clay and wood.

13. An appliance according to claim 1, wherein the spatula is contoured to conform to the body.

14. An appliance according to claim 1, wherein the spatula includes an upper surface for receiving the wax, and a lower surface for positioning on the body, and further comprising an adhesive positioned on the lower surface.

15. A hair removal appliance for aiding in the removal of a hardened wax from a body, comprising:
(a) an outer member defining an interior area, and having a first end curved upward in relation to the rest of the outer member to form a handle for being grasped by a user and a second substantially flat end for being positioned on the body; and
(b) an inner member connected to the outer member and positioned within the interior area defined by the outer member, wherein the inner member and the second end of the outer member define an opening for receiving liquid wax and allowing the wax to harden whereby the wax becomes attached to the appliance and removal of the appliance from the body simultaneously removes the wax from the body.

16. An appliance according to claim 15, wherein the outer member is substantially oval shaped.

17. A method of removing hair from a body, comprising the steps of:
(a) providing an appliance comprising a handle and a spatula having at least one cavity formed therein;
(b) positioning the appliance such that the spatula lies on the body and is adjacent a desired area of the body from which hair is desired to be removed;
(c) applying a liquid wax to the desired area of the body and the spatula;
(d) allowing a portion of the liquid wax on the spatula to be received within the cavity and harden therein; and
(e) after the wax has hardened, removing the appliance from the body whereby the hardened wax and hair attached to the wax are removed from the body.

18. A method according to claim 17, wherein the step of removing the appliance comprises grasping the handle and pulling the appliance away from the body.

19. A method according to claim 17, wherein the step of positioning the appliance further comprises positioning the appliance such that the spatula covers an area of the body on which hair is to remain.

20. A method according to claim 17, further comprising the steps of a user grasping the handle with a first hand, and maintaining the skin taut with a second hand.
